(19) United States
(12) Patent Application Publication
(10) Pub. No.: US 2007/0262704 A1
(43) Pub. Date: Nov. 15, 2007

(54) Carbazole-based Compounds and Their Application

(75) Inventors: Ming-Han Tsai, Taipei (TW); Hao-Wu Lin, Taipei (TW); Hai-Ching Su, Taipei (TW); Chung-Chih Wu, Taipei (TW); Fu-Chuan Fang, Kaohsiung County (TW); Yuan-Li Liao, Taipei (TW); Ken-Tsung Wong, Luzhou City (TW); Chih-I Wu, Taipei (TW); Shih-Feng Chiu, Pingtung City (TW)

Correspondence Address: WPAT, PC, 7225 BEVERLY ST., ANNANDALE, VA, 22003, US

(73) Assignee: Ken-Tsung Wong, Taipei County (TW)

(21) Appl. No.: 11/749,133

(22) Filed: May 15, 2007

Related U.S. Application Data
(60) Provisional application No. 60/800,019, filed on May 15, 2006.

(51) Int. Cl. H01J 1/62; C07F 7/02; C07D 209/82

(52) U.S. Cl. 313/503; 313/504; 548/406; 548/444

(57) ABSTRACT

The present invention discloses a carbazole-based compound with a general formula as following: 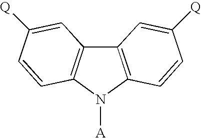 , wherein Q is a non-conjugate moiety, A comprises one of the following group: aryl moiety, hetero cycle, multiple fused ring, multiple fused ring with hetero atom(s). In addition, the present invention discloses a method for forming the carbazole-based compound.

### BACKGROUND OF THE INVENTION

1. Field of the Invention

The present invention is generally related to carbazole-based compounds, and more particularly to carbazole-based compounds which can be used as host materials in light-emitting devices.

2. Description of the Prior Art

Recent development of efficient phosphorescent emitters containing transition metals renders possible harvesting both electro-generated singlet and triplet excitons for emission from organic light-emitting devices (OLEDs) and realizing nearly 100% internal quantum efficiencies of electroluminescence (EL). In phosphorescent OLEDs, to reduce quenching associated with relatively long excited-state lifetimes of triplet emitters and triplet-triplet annihilation etc., triplet emitters are normally used as emitting guests in a host material. Effective host materials are thus of equal importance for efficient phosphorescent OLEDs. For efficient electrophosphorescence from triplet guests, it is essential that the triplet level of the host be larger than that of the triplet emitter to prevent reverse energy transfer from the guest back to the host and to effectively confine triplet excitons on guest molecules. Such a requirement becomes particularly challenging when blue electrophosphorescence is of interest, in which the conjugation in the host molecules must be extremely confined to achieve a triplet energy level larger than photon energies of blue light (i.e. ≧2.7 eV). As such, host materials reported for blue electrophosphorescence have thus far been rare and among them, materials based on the carbazole moiety are the only electrically active systems that had been spotted to meet such a requirement.

For molecules to form morphologically stable and uniform amorphous films with typical processing techniques, the molecule size must be extended beyond one carbazole monomer to obtain bulky and steric molecular configurations. Since conjugation beyond one carbazole would result in substantial reduction of the triplet energy, thus far there exist only a very limited number of carbazole-based compounds suitable for blue electrophosphorescence (i.e. having both large enough triplet energies and acceptable morphological stability). These rare cases in general involve connecting the carbazole moieties through its 9 position (i.e. the nitrogen atom) to a central linkage with even more limited conjugation (e.g. benzene or phenylsilanes), since such a connection appears to have little effect on conjugation. One distinguished example of such compounds is 1,3-bis(9-carbazolyl)benzene (hereinafter named as mCP, inset of FIG. 1(b)). Although such a molecular configuration has proved successful in improving morphological stability, one notices that all these materials have left unprotected their highly electrochemically active sites (C3 and C6 of carbazoles), which may cause issues in electrochemical stability. In light of the above-mentioned matter, it is required to develop a novel host material in OLEDs to obtain electrochemical stability and higher efficiency.

### SUMMARY OF THE INVENTION

According to the above, the present invention provides a new carbazole-based compound to fulfill the requirements of this industry.

One object of the present invention is to employ a novel molecular design strategy of retaining the large triplet energy of carbazole yet enhancing morphological stability by non-conjugated substitution of its C3 and C6 with bulky and large-gap moieties (i.e. triphenylsilyl groups). In addition, with such a configuration, the electrochemically active sites of carbazole are blocked, giving the compounds an extra electrochemical stability. According to the above, the present invention does have the economic advantages for industrial applications.

According to above-mentioned objectives, the present invention discloses a carbazole-based compound with a general formula as following: 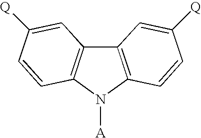
, wherein Q is a non-conjugate moiety, A comprises one of the following group: aryl moiety, hetero cycle, multiple fused ring, multiple fused ring with hetero atom(s). In addition, the present invention discloses a method for forming the carbazole-based compound.

### BRIEF DESCRIPTION OF THE DRAWINGS

FIG. 1 shows repeated cyclic voltammograms of (a) CzSi (5 cycles), and (b) mCP (5 cycles). Inset of (a) is the optimized molecular structure of CzSi and inset of (b) is the molecular structure of mCP;

FIG. 2 shows room-temperature absorption, fluorescence and 77 K phosphorescence spectra of CzSi in toluene (1×10⁻⁵ M), in comparison with those of the unsubstituted carbazole monomer in toluene;

FIG. 3 shows PL spectra of a CzSi film doped with 8 wt. % of FIrpic at the room temperature and 25 K, along with the EL spectrum of the device. Inset: PL intensity of the FIrpic-doped CzSi film as a function of temperature;

FIG. 4 shows chemical structures of (a) TCTA, (b) FIrpic and (c) TAZ. (d) Energy levels of related compounds in thin films;

FIG. 5 shows (a) I-V-L characteristics, and (b) external EL quantum efficiency/power efficiency vs. current density of the device;

FIG. 6 shows the detected characteristics of the devices respectively with CzSi, CzC, and CzCSi as the host material in the light-emitting layer; and

FIG. 7 shows EL spectra of the devices with respectively with CzSi, CzC, and CzCSi as the host material in the light-emitting layer.

### DESCRIPTION OF THE PREFERRED EMBODIMENTS

What is probed into the invention is carbazole-based compound and their application. Detail descriptions of the structure and elements will be provided in the following in order to make the invention thoroughly understood. Obviously, the application of the invention is not confined to specific details familiar to those who are skilled in the art. On the other hand, the common structures and elements that are known to everyone are not described in details to avoid unnecessary limits of the invention. Some preferred embodiments of the present invention will now be described in greater detail in the following. However, it should be recognized that the present invention can be practiced in a wide range of other embodiments besides those explicitly described, that is, this invention can also be applied extensively to other embodiments, and the scope of the present invention is expressly not limited except as specified in the accompanying claims.

The first embodiment of the present invention discloses a carbazole-based compound with a general formula as following: 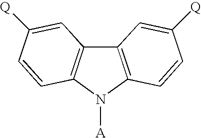
, wherein Q is a non-pi-conjugated moiety. A comprises one of the following group: aryl moiety, hetero cycle, multiple fused ring, multiple fused ring with hetero atom(s). Additionally, the glass transition temperature of the carbazole-based compound is equal to or higher than 100° C. Furthermore, the compound can be used as host material in organic electroluminescence devices.

In a preferred example of this embodiment, the carbazole-based compound has a general formula as following: 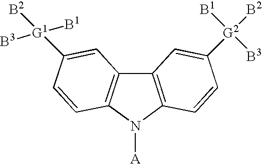
, wherein G¹ and G² are identical or different, G¹ and G² are independently selected from C, Si. B¹, B² and B³ are identical or different, and B¹, B² and B³ are independently selected from the group consisting of: linear alkyl, branched alkyl, cyclic alkyl, aryl moiety, hetero cycle, multiple fused ring, multiple fused ring with hetero atom(s), and alkyl with at least one substituent of alkene or alkyne or carbamates.

In another preferred example of this embodiment, the carbazole-based compound has a general formula as following: 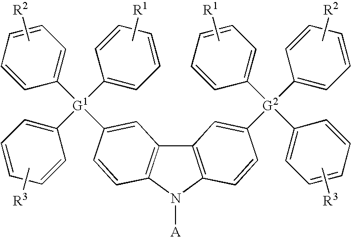
wherein G¹ and G² are identical or different, G¹ and G² are independently selected from C, Si. R¹, R² and R³ are identical or different, and R¹, R² and R³ are independently selected from the group consisting of: hydrogen atom, linear alkyl, branched alkyl, cyclic alkyl, aryl moiety, hetero cycle, multiple fused ring, multiple fused ring with hetero atom(s), and alkyl with at least one substituent of alkene or alkyne or carbamates.

### EXAMPLE 1

### 9-(4-tert-butylphenyl)-3,6-bis(triphenylsilyl)-9H-carbazole (Hereinafter Referred to as CzSi) and Synthesis Thereof

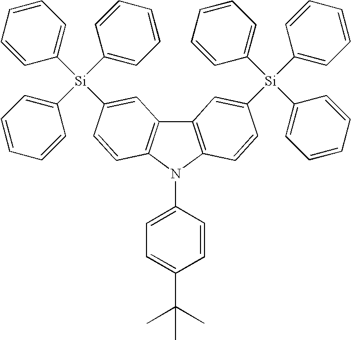

CzSi: 9-(4-tert-butylphenyl)-3,6-dibromo-carbazole (1.8 g, 4 mmol) in THF (150 mL) were treated with n-BuLi (7.5 ml, 12 mmol) at −78° C. and quenched with a solution of chlorotriphenylsilane (3.54 g, 12 mmol) in THF (50 mL). The desired product was purified by column chromatography, eluting with CHCl₃/Hexane (1/4) to provide the product as a white solid. (1.2 g, 35%).

CzSi: m.p. 354° C. (DSC); IR (neat) ν 2960, 2359, 1681, 1428, 1260, 1025, 801, 699, 512 cm⁻¹; ¹H NMR (CDCl₃, 400 MHz) δ 8.21 (s, 2H), 7.61-7.55 (m, 16H), 7.49-7.41 (m, 2H), 7.44-7.40 (m, 8H), 7.37-7.34 (m, 12H) 1.42 (s, 9H); ¹³C NMR (CDCl₃, 100 MHz) δ 150.1, 141.5, 136.0, 134.4, 134.0, 133.4, 129.0, 128.5, 127.4, 126.3, 126.1, 123.5, 122.7, 109.5, 35.1, 31.7; MS (m/z, FAB⁺) 815 (4); HRMS (m/z, FAB⁺) Calcd for C₅₈H₄₉NSi₂ 815.3404, found 815.3403; Anal. Calcd. For C₅₈H₄₉NSi₂: C, 85.35; H, 6.05. Found: C, 85.10; H, 6.07.

### EXAMPLE 2

### 3,6-bis(triphenylmethyl)-9-(4- tert-Butylphenyl)-carbazole (Hereinafter Referred to as CzC) and Synthesis Thereof

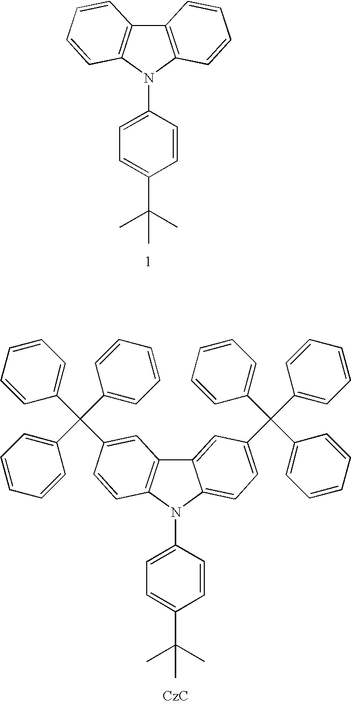

CzC: 9-(4-tert-Butylphenyl)carbazole (1) (2.99 g, 10 mmol) and triphenyl methanol (5.47 g, 21 mmol) was dissolved in CH₂Cl₂ (70 mL), Eaton's reagent (7.7 wt % P₂O₅ in CH3SO3H) was added dropwise at 25° C. under a nitrogen atmosphere. After 24 h, the precipitate was filtered and washed with water (50 mL×2) and dilute aqueous sodium bicarbonate solution (×1). The crude in acetone was refluxed for 1 h, and refluxed for another 1 h in CH₂Cl₂. The white powder were filtered and dried: yield 6.6 g (84%).

CzC: mp 372˜374° C. (DSC); ¹H NMR (CDCl₃, 400 MHz) δ 7.79 (d, J=1.6 Hz, 2H), 7.53 (dd, J=8.8 Hz, 2H), 7.47 (dd, J=8.8 Hz, 2H), 7.30 (s, 1H), 7.30˜7.26 (m, 4H), 7.26-7.16 (m, 27H), 7.12 (dd, J=8.8, 2.0 Hz, 2H), 1.40 (s, 9H); ¹³C NMR (CDCl₃, 400 MHz) δ 150.0, 147.1, 139.0, 138.4, 134.8, 131.1, 130.4, 127.2, 126.4, 126.0, 125.6, 122.3, 121.3, 108.9, 64.9, 34.8, 31.5; MS(m/z, FAB⁺) 783 (0.41); HRMS (M⁺, FAB⁺) Calcd. for C₆₀H₄₉N 783.3865, Found 783.3871.

### EXAMPLE 3

### 3-triphenylmethyl-6-triphenylsilyl-9-(4- tert-Butylphenyl)-carbazole (Hereinafter Referred to as CzCSi) and Synthesis Thereof

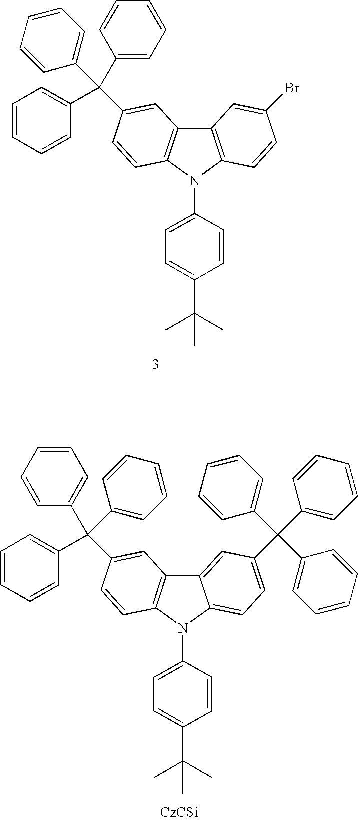

CzCSi: n-BuLi (5.5 mmol, 3.4 ml of 1.6 molL⁻¹ solution) was dropwised slowly to a degassed THF solution (50 mL) of 3-bromo-6-tripheylmethyl-9-(4-tert-Butylphenyl)-carbazol (3) (3.1 g, 5 mmol) at −78° C. After stirring for 1 h at −78° C., chlorotriphenylsilane (1.62 g, 5.5 mmol) in THF (30 mL) was added in one portion. The resulting mixture was immediately warmed to room temperatrure. The precipitate was filtered and washed with hexane (50 mL) to provide the white powder: yield 2.4 g (60%).

CzCSi: mp 357˜360° C. (DSC); ¹H NMR (CDCl₃, 400 MHz) δ 8.11 (s, 1H), 7.88 (d, J=1.2 Hz, 1H), 7.61-7.59 (m, 5H), 7.54 (t, 4H), 7.48 (s, 2H), 7.46 (s, 1H), 7.42 (dd, J=7.8, 5.4 Hz, 5H), 7.36 (t, J=7 Hz, 7H), 7.3 (s, 1H), 7.28 (d, J=5.6 Hz, 3H), 7.24-7.15 (m, 10H), 1.40 (s, 9H); ¹³C NMR (CDCl₃, 400 MHz) δ 150.2, 147.1, 142.0, 138.9, 138.7, 136.3, 134.8, 134.6, 133.4, 131.1, 130.6, 129.3, 128.6, 127.7, 127.2, 126.5, 126.1, 125.7, 123.3, 123.2, 122.0, 121.6, 109.7, 108.9, 64.9, 34.8, 31.5; MS(m/z, FAB⁺) 799 (100)

### EXAMPLE 4

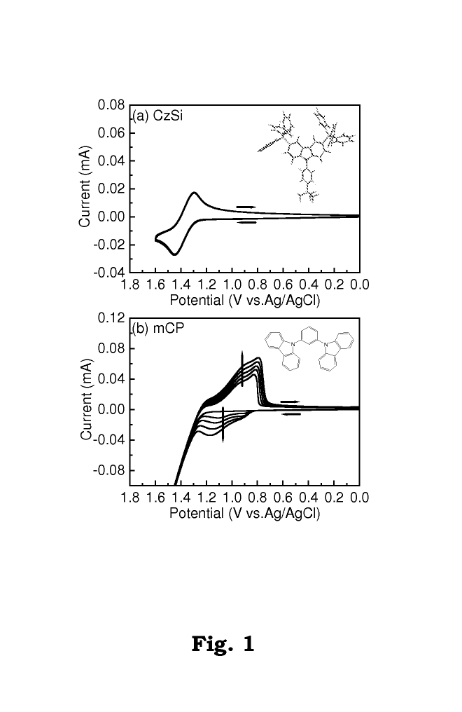

As shown in the optimized molecular structure of CzSi (inset of FIG. 1(a)), linking triphenylsilyl groups to C3 and C6 of carbazole renders the molecule rather steric, rigid, and bulky. Such a molecular configuration is strongly beneficial to the thermal stability, as indicated by the high decomposition temperature (T_{d}, corresponding to 5% weight loss in the thermogravimetric analysis) of 392° C. and the rather high glass transition temperature (T_{g}) of 131° C. determined by differential scanning calorimetry. Such a T_{g} is more than double of mCP's T_{g} (<60° C.). With a high T_{g} and thus resistance against crystallization, CzSi is able to form homogeneous and stable amorphous films by thermal evaporation.

The electrochemical properties of CzSi are investigated by cyclic voltammetry and are shown in FIG. 1(a). Synthesized compounds were subject to purification by temperature-gradient sublimation in high vacuum before use in subsequent studies. Cyclic voltammetry (CV) was performed at a scan rate of 100 mV/s using the glass electrode as the working electrode and Ag/AgCl as a reference electrode. Oxidation CV was performed in CH₂Cl₂ with 0.1 M of nBu₄NPF₆ as a supporting electrolyte, and then a reversible oxidation process [E_{1/2}=1.37 V (vs. Ag/AgCl)] was observed. In contrast, without blocking the electrochemically active sites (C3 and C6 of carbazoles), the oxidation process of mCP is not reversible, with the oxidation potential gradually shifting to lower potentials and the current increasing during repeated CV scans (FIG. 1(b)). As revealed in literature, such characteristics are signatures of electrochemical polymerization of carbazoles through the active C3 and C6 sites. These electrochemical results indicate that in addition to the morphological benefits, the introduction of triphenylsilyl substitutions brings enhanced electrochemical stability as they block the electrochemically active sites (C3 and C6) of carbazole.

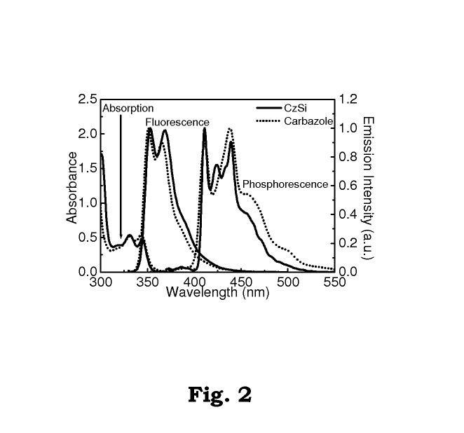

FIG. 2 shows the room-temperature absorption, fluorescence and 77 K phosphorescence spectra of CzSi, which are nearly identical to those of the unsubstituted carbazole monomer (FIG. 2) and thus can be unambiguously attributed to the lowest π-π* transition of the central carbazole chromophore in CzSi. The tetrahedral Si therefore serves as an effective spacer blocking the π-conjugation of the carbazole core from extending to the peripheral substitution. Furthermore, it is noticed that the phenyl substitution at the 9 position of carbazole has no effect on these photophysical characteristics either, consistent with previous observations. By taking the highest-energy peak of phosphorescence as the transition energy of T₁→S₀, which corresponds to the vibronic 0-0 transition between these two electronic states, the triplet energy of CzSi is estimated to be as high as 3.02 eV, same as that of the unsubstituted cabazole.

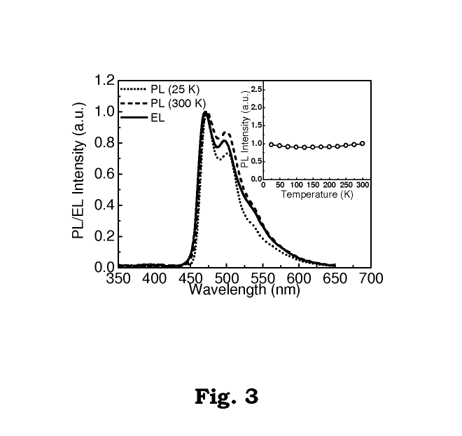

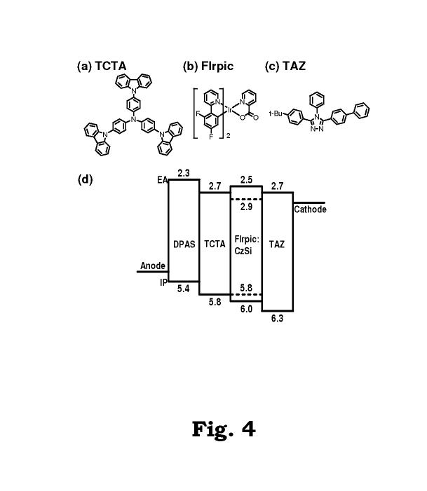

With the high triplet energy, CzSi is considered as a promising host for blue electrophosphorescence. The most reliable means for testing the effectiveness of a host for a phosphorescent dopant is to perform temperature-dependent photoluminescence (PL) of the host-guest system. In general, if the host-to-guest energy transfer is endothermic (and thus thermally activated) and the exciton confinement is not effective enough, at low temperatures the host-to-guest energy transfer would be substantially retarded and the back energy transfer might occur. As such, one may observe fluorescence and/or phosphorescence from the host and substantial reduction of the overall luminescence efficiency due to larger probability of relaxation from the less efficient host molecules. FIG. 3 shows temperature dependence of PL of a CzSi film doped with 8 wt. % of the typical blue phosphorescent dopant iridium(III)bis [4,6-difluorophenyl]-pyridinato-N,C²′]picolinate (FIrpic, FIG. 4). For the whole temperature range of 25-300 K investigated, the FIrpic-doped CzSi shows only emission of FIrpic and no emission of CzSi (either fluorescence or phosphorescence) could be observed (FIG. 3). Furthermore, the PL intensity is rather independent of the temperature (inset of FIG. 3). These results indicate efficient exothermic energy transfer from CzSi to FIrpic and effective confinement of triplet excitons on dopants. Along with the high PL quantum yield of 92% for the FIrpic-doped CzSi, the temperature-independent PL also suggests thermally activated nonradiative decay processes in the present host-guest system are much weaker than FIrpic phosphorescence.

PL of organic thin films was measured using a CCD spectragraph and the 325-nm line of the He—Cd laser as the excitation source. During temperature-dependent PL measurements, the samples were mounted in a vacuum chamber equipped with a temperature controller. For determining PL quantum yields, the samples were mounted in a calibrated integrating sphere coupled to the CCD spectragraph. By comparing the spectral intensities of the excitation laser and the PL emission, PL quantum yields were determined. Phosphorescence of CzSi was measured at 77° K (the liquid nitrogen temperature) using a 5-ms delay time between the excitation with a microsecond flash lamp and the measurement.

The second embodiment of the present invention discloses an organic light emitting device comprising a multilayer structure for producing electroluminescence, wherein the multilayer structure comprises: a substrate, an anode layer, a first hole transporting layer comprising 4,4′-bis[N-(1-naphthyl)-N-phenyl-amino]biphenyl (α-NPD) or 2,2′-bis (N,N-disubstituted amine)-9,9′-spirobifluorene, a second hole transporting layer, an emitting layer comprising a host material and a guest material, wherein the host material comprises carbazole-based compound, an electron transporting layer, and a cathode layer. Furthermore, the general formula of the 2,2′-bis (N,N-disubstituted amine)-9,9′-spirobifluorene is as following: 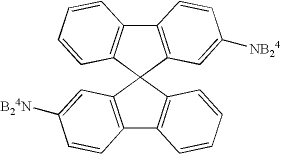
, wherein B⁴ is selected from the group consisting of: linear alkyl, branched alkyl, cyclic alkyl, aryl moiety, hetero cycle, multiple fused ring, multiple fused ring with hetero atom(s), and alkyl with at least one substituent of alkene or alkyne or carbamates. Moreover, the general formula of the carbazole-based compound is as following: 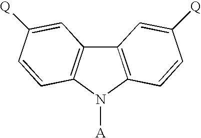
, wherein Q of the carbazole-based compound is a non-conjugate moiety, A comprises one of the following group: aryl moiety, hetero cycle, multiple fused ring, multiple fused ring with hetero atom(s).

In this embodiment, the chemical structure of one preferred example of the 2,2′-bis (N,N-disubstituted amine)-9,9′-spirobifluorene is 2,2′-Bis(diphenylamino)-9,9′-spirobifluorene (hereinafter referred to as DPAS) as the following: 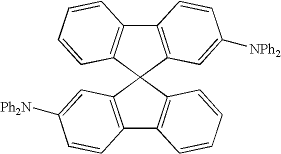
The second hole transporting layer can comprise 4,4′,4″-tri(N-carbazolyl) triphenylamine (TCTA). The guest material comprises iridium(III)bis[4,6-difluorophenyl]-pyridinato-N,C²′]picolinate (FIrpic). Additionally, the carbazole-based compound is described in the first embodiment of this invention.

### EXAMPLE 5

CzSi has been subjected to electrophosphorescence studies. The OLEDs were fabricated on glass substrates with the typical structure of multiple organic layers sandwiched between the bottom indium tin oxide (ITO) anode and the top metal cathode (Al). The PEDT:PSS layer was prepared by spin coating, and other material layers were deposited by vacuum evaporation in a vacuum chamber with a base pressure of <10⁻⁶ torr. The deposition system permits the fabrication of the complete device structure in a single vacuum pump-down without breaking vacuum. The deposition rate of organic layers was kept at ˜0.2 nm/s. The active area of the device is 2×2 mm², as defined by the shadow mask for cathode deposition. The device structure used was ITO/PEDT:PSS (˜300 Å)/DPAS or α-NPD (175 Å)/TCTA (25 Å)/CzSi doped with 8 wt. % FIrpic (250 Å)/TAZ (500 Å)/LiF (5 Å)/Al (1500 Å), where the conducting polymer polyethylene dioxythiophene/polystyrene sulphonate (PEDT:PSS) was used as the hole-injection layer, DPAS, 4,4′-bis[N-(1-naphthyl)-N-phenyl-amino]biphenyl (α-NPD), and 4,4′,4″-tri(N-carbazolyl) triphenylamine (TCTA) as the hole-transport layers, CzSi with a nearly optimized concentration (8 wt. %) of FIrpic as the emitting layer, 3-(4-biphenylyl)-4-phenyl-5-(4-tert-butylphenyl)-1,2,4- triazole (TAZ) as the electron-transport layer, and LiF as the electron-injection layer. Chemical structures of related compounds and their energy levels are shown in FIG. 4, in which ionization potentials (IP) of molecular compounds in films were determined by our own measurements with ultraviolet photoemission spectroscopy while IP of FIrpic was taken from the literature. Electron affinities (EA) of all compounds were derived by subtracting IP's with optical energy gaps.

IP's of thin films of organic compounds were measured by ultraviolet photoemission spectroscopy (UPS). The deposition and the UPS measurements of thin-film samples were performed in two interconnected ultra-high vacuum chambers. Organic thin films were deposited on gold-coated silicon substrates by thermal evaporation in the deposition chamber, and then transferred in situ to the analysis chamber. In the analysis chamber with base pressure less than 1×10⁻¹⁰ Torr, UPS was carried out using the He I (21.22 eV) and He II (40.8 eV) photon lines and the double-pass cylindrical mirror analyzer to measure energy spectrum of photo-excited electrons. The overall resolution of the UPS measurement is about 0.15 eV. The energy scale of UPS spectra is referenced to the Fermi level of the system, which is measured on the gold surface before deposition of organic thin films. IP's of molecular films can be deduced from the energy difference between the HOMO level and the vacuum level (inferred from the low-energy onset of the UPS spectrum).

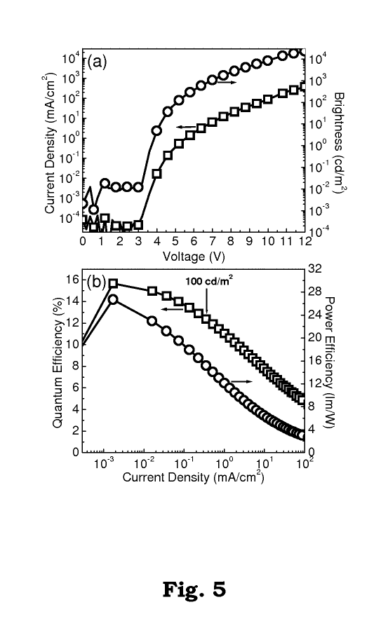

As shown in FIG. 3, EL of the device using the DPAS/TCTA hole-transport layers is identical to PL of FIrpic-doped CzSi, indicating the effectiveness of the present device structure in injecting both holes and electrons into the emitting layer. I-V-L characteristics, external EL quantum efficiency and power efficiency of the device are shown in FIG. 5(a) and FIG. 5(b). The current-voltage-brightness (I-V-L) characterization of the light-emitting devices was performed with a source-measurement unit (SMU) and a Si photodiode calibrated with Photo Research PR650. EL spectra of devices were collected by a calibrated CCD spectragraph. The present device exhibits a rather low turn-on voltage of ˜3 V (defined as the voltage where EL becomes detectable) and a low operation voltage (100 cd/m² at 5V), as shown in FIG. 5(a). A high external EL quantum efficiency of 15.7% photon/electron (30.6 cd/A, maximum) and a maximum brightness as high as ˜59000 cd/m² (at 14.5 V) were observed. High quantum efficiency along with low voltage gives maximal power efficiency of 26.7 lm/W. Similar device performances were also obtained by using the α-NPD/TCTA hole-transport layers, yet DPAS has a higher Tg (and thus thermal stability) than α-NPD (˜110° C. vs. ˜90° C.). In addition to achieving an external quantum efficiency among the highest ever reported for blue electrophosphorescence, the power efficiency of the present device is also nearly double of the highest values previously reported for blue electrophosphorescence (˜14 lm/W). These improved characteristics may, in part, be attributed to the judicious use of two hole-transport layers (DPAS/TCTA or α-NPD/TCTA) with a stepwise increase in IP's to match IP of CzSi, and choice of the electron-transport layer (TAZ). Using only single hole-transport layer of α-NPD, DPAS or TCTA results in lower efficiency and higher voltage. Adopting other electron-transport and hole-blocking layers widely used in phosphorescent OLEDs, such as BCP (2,9-dimethyl-4,7-diphenyl-1,10-phenanthroline) or TPBI (2,2′,2″-(1,3,5-benzenetriyl)tris[1-phenyl-1H-benzimidazole]), also substantially reduces the device efficiency. Efficiency roll-off at higher currents, which is typical in phosphorescent OLEDs and may be associated with triplet-triplet annihilation, is also observed here, yet at the practical brightness of 100 cd/M² (5 V, 0.36 mA/cm²), the efficiencies remain above 12%, 24 cd/A and 16 lm/W.

Finally, to gain further insights of charge transport and emission mechanisms in the devices, three testing devices with doping only a portion of the 25-nm CzSi emitting layer (device A: doping 8 nm next to DPAS/TCTA; device B: doping 8 nm at the center; device C: doping 8 nm next to TAZ) were also fabricated and tested. Although all three devices show emission dominantly from FIrpic, devices A and B show much lower quantum efficiencies (5% and 6%, respectively) than device C (14%). The results indicate the EL emission mainly takes place near the CzSi/TAZ interface, where electrons inject either onto CzSi or directly onto FIrpic. This in turn suggests the hole-transport capability of CzSi and feasible hole injection from DPAS/TCTA onto CzSi.

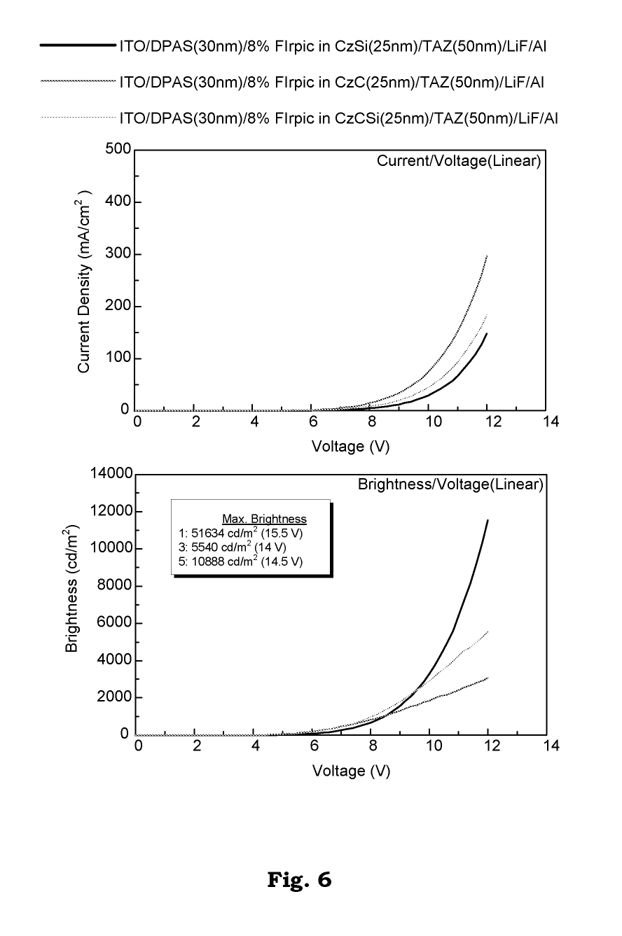

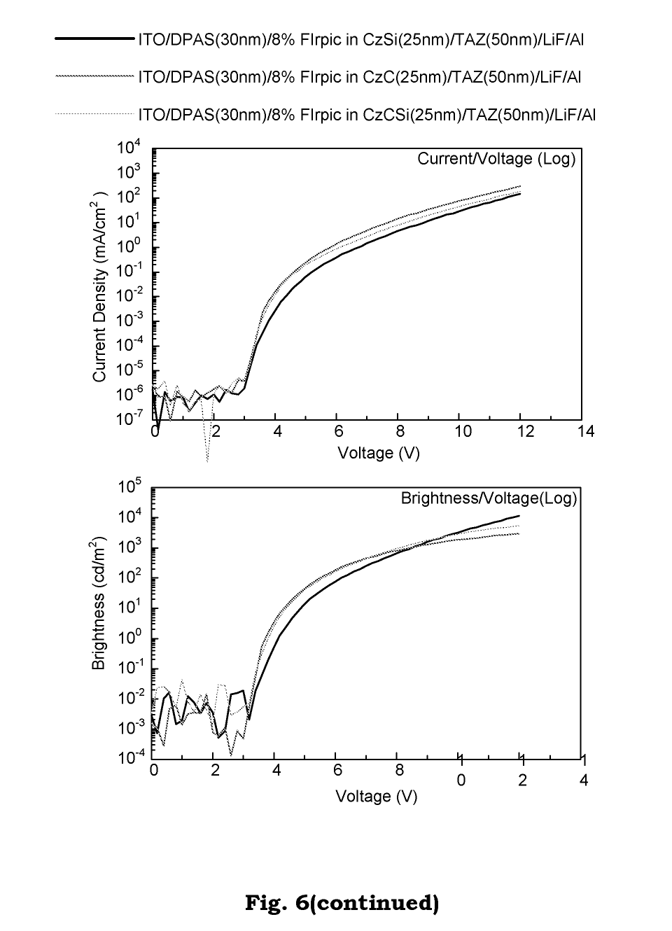

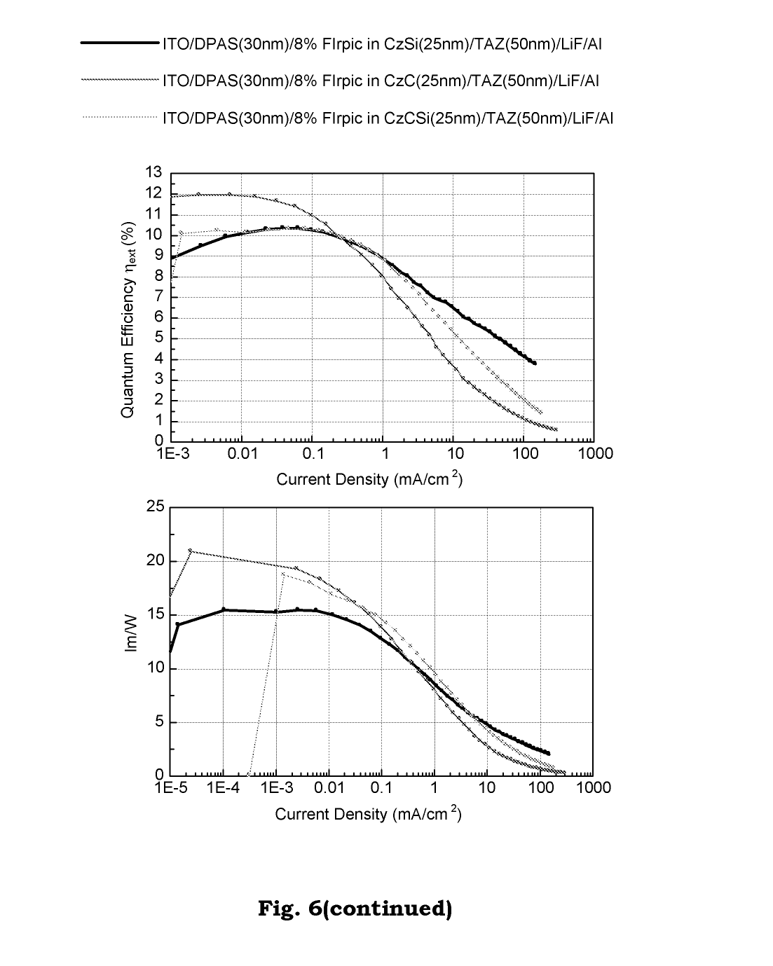

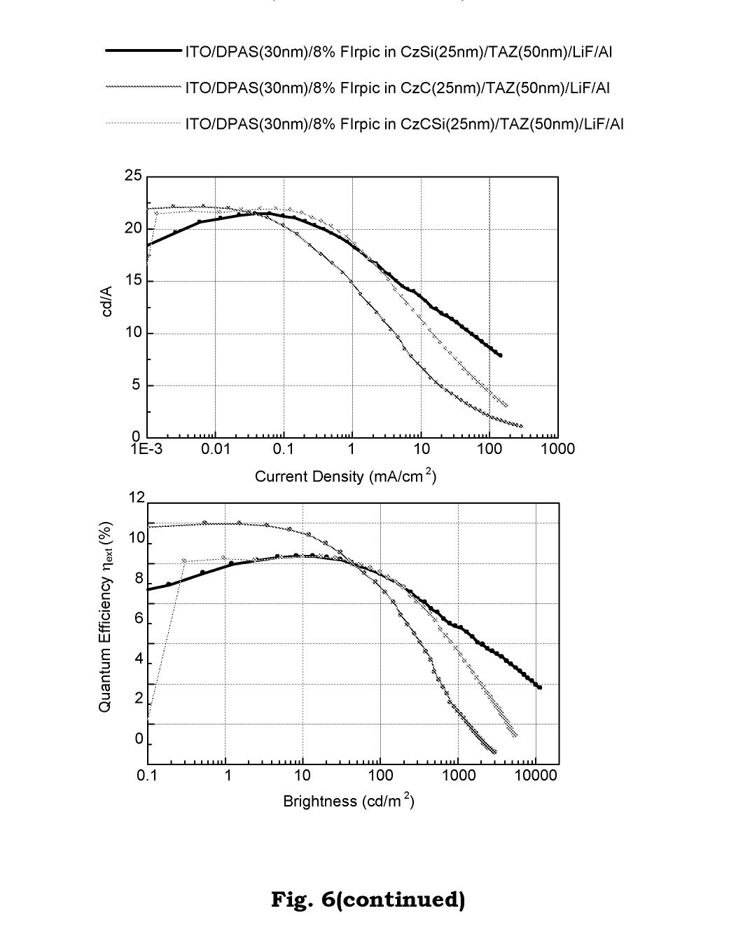

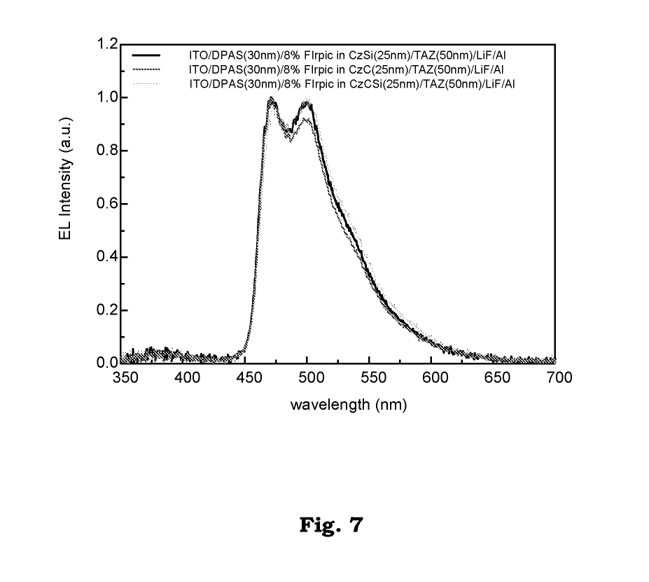

Moreover, as shown in FIG. 6 and FIG. 7, CzSi, CzC, CzCSi have been individually subjected to electrophosphorescence studies. Using the mentioned method, the light-emitting devices having the following structures were produced:
ITO/DPAS (300 Å)/8 wt. % FIrpic in CzSi (250 Å)/TAZ (500 Å)/LiF (5 Å)/Al (1500 Å)
ITO/DPAS (300 Å)/8 wt. % FIrpic in CzC (250 Å)/TAZ (500 Å)/LiF (5 Å)/Al (1500 Å)
ITO/DPAS (300 Å)/8 wt. % FIrpic in CzCSi (250 Å)/TAZ (500 Å)/LiF (5 Å)/Al (1500 Å)

And, some detected spectra of the above-mentioned device are shown in FIG. 6 and FIG. 7.

The third embodiment of the present invention discloses a method for forming a carbazole-based compound. As shown in Scheme 1, first a dihalo derivative of carbazole, a halo-trisubstituted silane and a Lewis base as auxiliary are provided, and then a substitution reaction is performed to react the dihalo derivative of carbazole with the halo-trisubstituted silane to produce the carbazole-based compound. X¹ and X² are independently selected from the group consisting of: Cl, Br and I.

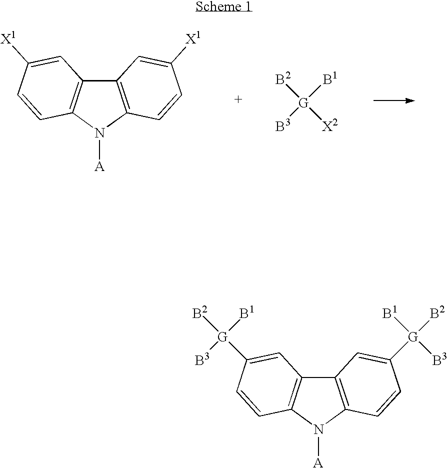

In this embodiment, the dihalo derivative of carbazole has a general formula as following: 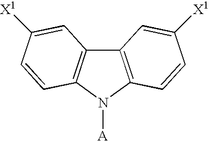
wherein A comprises one of the following group: aryl moiety, hetero cycle, multiple fused ring, multiple fused ring with hetero atom(s). The halo-trisubstituted silane has a general formula as following: 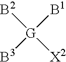
, wherein G comprises one of the following group: C, Si. Additionally, B¹, B² and B³ are identical or different, and B¹, B² and B³ are independently selected from the group consisting of: linear alkyl, branched alkyl, cyclic alkyl, aryl moiety, hetero cycle, multiple fused ring, multiple fused ring with hetero atom(s), and alkyl with at least one substituent of alkene or alkyne or carbamates. Moreover, the carbazole-based compound has a general formula as following: 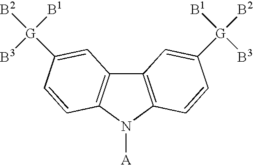
wherein A, G, B¹, B² and B³ are described above.

### EXAMPLE 6

The synthetic pathway of the targeted compound, 9-(4-tert-butylphenyl)-3,6-bis(triphenylsilyl)-9H-carbazole (hereinafter named as CzSi), is shown in Scheme 2. 9-(4-tert-butylphenyl)-3,6-dibromo-carbazole (1.8 g, 4 mmol) in THF (150 mL) were treated with n-BuLi (7.5 ml, 12 mmol) at −78° C. and quenched with a solution of chlorotriphenylsilane (3.54 g, 12 mmol) in THF (50 mL). The desired product was purified by column chromatography, eluting with CHCl₃/Hexane (1/4) to provide the product as a white solid. (1.2 g, 35%);

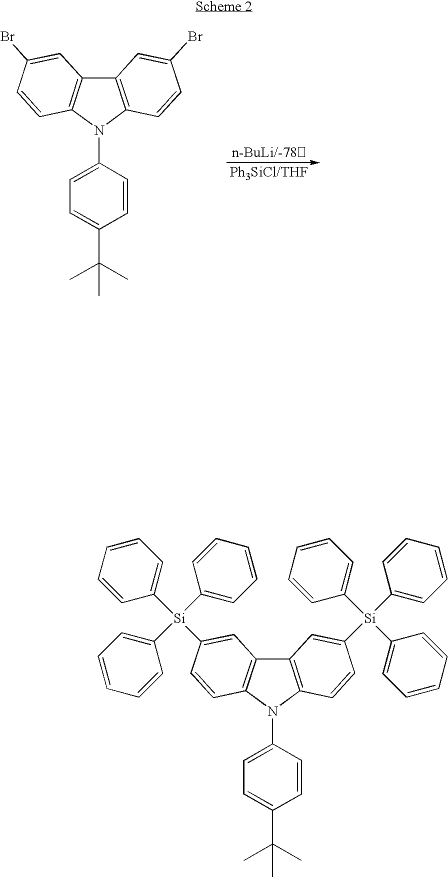

In the embodiments, 3,6-bissubstituted carbazole is provided as an effective host material for blue electrophosphorescence. By non-conjugated substitution of the electrochemically active C3 and C6 sites of carbazole with the steric, bulky and large-gap triphenylsilyls, the new compound retains the large triplet energy of carbazole yet exhibits much enhanced morphological stability and electrochemical stability in comparison with previous carbazole-based host materials. For example, when CzSi is used as host material, blue phosphorescent OLEDs having high efficiencies up to 16%, 30.6 cd/A and 26.7 lm/W are demonstrated. Although mainly blue devices are reported here, such a large-gap host materials may also be of use for green and red phosphorescent devices. Indeed, our preliminary results on green phosphorescent devices using the same device structure have shown comparable quantum efficiencies.

To sum up, the present invention discloses a carbazole-based compound with a general formula as following: 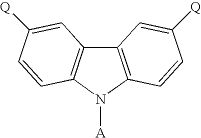
, wherein Q is a non-pi-conjugate moiety, A comprises one of the following group: aryl moiety, hetero cycle, multiple fused ring, multiple fused ring with hetero atom(s). In addition, the present invention discloses a method for forming the carbazole-based compound.

Obviously many modifications and variations are possible in light of the above teachings. It is therefore to be understood that within the scope of the appended claims the present invention can be practiced otherwise than as specifically described herein. Although specific embodiments have been illustrated and described herein, it is obvious to those skilled in the art that many modifications of the present invention may be made without departing from what is intended to be limited solely by the appended claims.

What is claimed is:

1. A carbazole-based compound with a general formula as following: 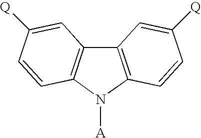
, wherein Q is a non-pi-conjugate moiety; and A comprises one of the following groups: aryl moiety, hetero cycle, multiple fused ring, multiple fused ring with hetero atom(s).

3. The carbazole-based compound as claimed in claim 1, wherein the glass transition temperature of the carbazole-based compound is equal to or higher than 100° C.

4. The carbazole-based compound as claimed in claim 1, wherein the carbazole-based compound has a general formula as following: 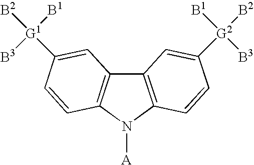
wherein G¹ and G² are identical or different, G¹ and G² are independently selected from C, Si; and B¹, B² and B³ are either identical or different from one another(redundant, may as well be deleted), and B¹, B² and B³ are independently selected from the group consisting of: linear alkyl, branched alkyl, cyclic alkyl, aryl moiety, hetero cycle, multiple fused ring, multiple fused ring with hetero atom(s), and alkyl with at least one substituent of alkene or alkyne or carbamates.

5. The carbazole-based compound as claimed in claim 1, wherein the carbazole-based compound has a general formula as following: 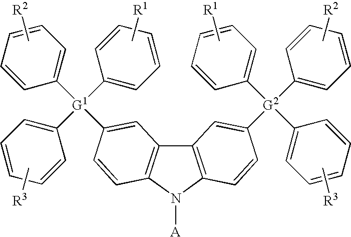
wherein G¹ and G² are identical or different, G¹ and G² are independently selected from C, Si; and R¹, R² and R³ are either identical or different form one another, and R¹, R² and R³ are independently selected from the group consisting of: hydrogen atom, alkoxyl group, NR2 (R is alkyl or aryl), linear alkyl, branched alkyl, cyclic alkyl, aryl moiety, hetero cycle, multiple fused ring, multiple fused ring with hetero atom(s), and alkyl with at least one substituent of alkene or alkyne or carbamates.

6. The carbazole-based compound as claimed in claim 1, wherein the chemical structure of the carbazole-based compound is as following:

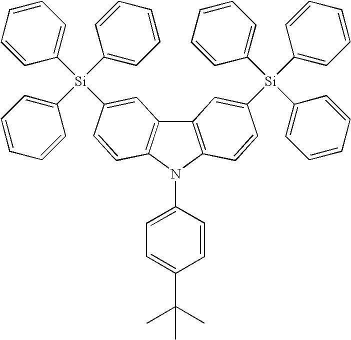

7. The compound as claimed in claim 1, wherein the carbazole-based compound is used in organic electroluminescent devices.

8. The compound as claimed in claim 1, wherein the carbazole-based compound is used as host material in organic electroluminescent devices.

9. A light-emitting device comprising a pair of electrodes and one or more organic layers disposed between said electrodes, said one or more organic layers comprising a light-emitting layer, wherein at least one of said one or more organic layer comprises a carbazole-based compound with a moiety represented by the following formula: 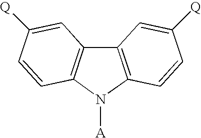
wherein Q is a non-pi-conjugate moiety, A comprises one of the following groups: aryl moiety, hetero cycle, multiple fused ring, multiple fused ring with hetero atom(s).

10. The light-emitting device as claimed in claim 9, wherein the carbazole-based compound has a general formula as following: 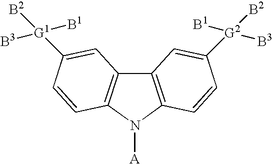
wherein G¹ and G² are identical or different, G¹ and G² are independently selected from C, Si; and B¹, B² and B³ are either identical or different from one another, and B¹, B² and B³ are independently selected from the group consisting of: linear alkyl, branched alkyl, cyclic alkyl, aryl moiety, hetero cycle, multiple fused ring, multiple fused ring with hetero atom(s), and alkyl with at least one substituent of alkene or alkyne or carbamates.

11. The device as claimed in claim 9, wherein the carbazole-based compound has a general formula as following: 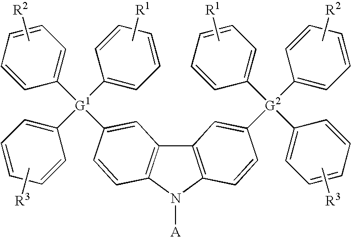
wherein G¹ and G² are identical or different, G¹ and G² are independently selected from C, Si; and R¹, R² and R³ are either identical or different from one another, and R¹, R² and R³ are independently selected from the group consisting of: hydrogen atom, linear alkyl, branched alkyl, cyclic alkyl, aryl moiety, hetero cycle, multiple fused ring, multiple fused ring with hetero atom(s), and alkyl with at least one substituent of alkene or alkyne or carbamates.

12. The light-emitting device as claimed in claim 9, wherein said carbazol-based compound is a host material in the light-emitting layer.

13. The light-emitting device as claimed in claim 9, wherein said light-emitting layer comprises iridium(III)bis[4,6-difluorophenyl]-pyridinato-N,C²′]picolinate (FIrpic) as a guest material.

14. The light-emitting device as claimed in claim 9, wherein said one or more organic layer further comprises a hole transport layer comprising 2,2′-Bis(diphenylamino)-9,9′-spirobifluorene:

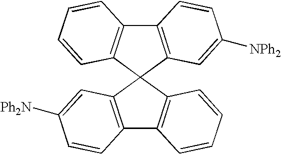

15. The light-emitting device as claimed in claim 14, wherein the hole transport layer further comprises 4,4′,4″-tri(N-carbazolyl) triphenylamine (TCTA).

16. The light-emitting device as claimed in claim 9, wherein the glass transition temperature of the carbazole-based compound is at or above 100° C.

17. The device as claimed in claim 9, wherein the chemical structure of the carbazole-based compound is as following: [chemical structure]
